(19) United States
(12) Patent Application Publication
(10) Pub. No.: US 2017/0002306 A1
(43) Pub. Date: Jan. 5, 2017

(54) Gas Recirculation System for an Incubated Controlled Environment

(71) Applicants: Michael Cecchi, Longboat Key, FL (US); Timothy Schimmel, Randolph, NJ (US); Monica Mezzezi, North Redington, FL (US); Jacques Cohen, New York, NY (US); Michael R. Cecchi, Longboat Key, FL (US)

(72) Inventors: Michael Cecchi, Longboat Key, FL (US); Timothy Schimmel, Randolph, NJ (US); Monica Mezzezi, North Redington, FL (US); Jacques Cohen, New York, NY (US); Michael R. Cecchi, Longboat Key, FL (US)

(21) Appl. No.: 14/789,591

(22) Filed: Jul. 1, 2015

(51) Int. Cl. C12M 1/00; C12M 1/34

(52) U.S. Cl. CPC C12M 29/24; C12M 41/14; C12M 41/34; C12M 23/40; C12M 29/04

(57) ABSTRACT

A gas recirculation system for an incubated controlled environment, the system comprising: a first mixing box; a second mixing box in fluid communication with the first mixing box; a manifold in fluid communication with the second mixing box; a plurality of incubation chambers in fluid communication with the manifold; a return manifold in fluid communication with the plurality of incubation chambers and in fluid communication with the first mixing box. A gas recirculation system for an incubated controlled environment, the system comprising: a first mixing box; a second mixing box in fluid communication with the first mixing box; an inlet port in fluid communication with the second mixing box; a plurality of incubation chamber inlet ports; an incubation chamber in fluid communication with the plurality of incubation chamber inlet ports; a plurality of incubation chamber outlet ports in fluid communication with the incubation chamber; a check valve in fluid communication with the plurality of incubation outlet ports, and in fluid communication with the second mixing box. A gas recirculation system for an incubated controlled environment, the system comprising: a pump; a filter in fluid communication with the pump; a sensor box in fluid communication with the filter; a pH sensor box in fluid communication with the sensor box, the pH sensor box comprising a container with embryo culture media and a pH sensor configured to determine the pH of the embryo culture media; a regulator in fluid communication with the pH sensor box; an embryo chamber in fluid communication with the regulator; a second pH sensor box in fluid communication with the regulator, the pH sensor box comprising a container with embryo culture media and a pH sensor configured to determine the pH of the embryo culture media. A gas recirculation system for an incubated controlled environment, the system comprising: a means for monitoring the O2 and CO2 concentration of gases in the incubated controlled environment; a means for injecting CO2, N2, and/or O2 into the incubated controlled environment via a mixing box; a means for adjusting the CO2, N2, and O2 levels in the incubated controlled environment. A gas recirculation system for an incubated controlled environment of claim 16, further comprising: a means for introducing CO2, N2, and/or O2 into the incubated environment in order to maintain about 5% of CO2; about 90% of N2; and/or about 5% of O2 in the incubated environment.

### CROSS-REFERENCES

This patent application claims priority to provisional patent application No. 62/020,048 filed on Jul. 2, 2014, by Michael Cecchi, et al, and titled: “Gas Recirculation System for an Incubated Controlled Environment” which provisional application is fully incorporated by reference herein.

### TECHNICAL FIELD

This invention relates to an apparatus and method for the long-term, uninterrupted, and culturing of embryos and biological specimens in a controlled incubated environment.

### BACKGROUND

Currently, incubators are produced in a ‘big box’ platform whereby specimens are cultures within a single large box, a single chamber, with a single source of incoming gases and single monitoring systems. The majority of these systems inject CO2 and N2, into these large boxes, through single ports for each gas. These current systems do not rely on recirculating the gas and only control the flow of incoming gases in an attempt to balance the internal gas mixtures and percentages. These large box incubators are cumbersome and difficult to maintain and do not maintain the balance of critical gases very well.

Other current incubators may be smaller and have a few compartments or chambers, and use a continuous stream of gases through these compartments. These incubators use either a premixed or a mixture of gases, from separate carbon dioxide and nitrogen tanks, which are then mixed and then used in the incubators. These other incubators generally do not constantly balance, monitor or recirculate the gases, and thereby may provide various mixtures at different times and may result in the using of a larger amount of gases, which may increases the cost of the incubators, and requires manpower to change and replenish the gas tanks. Current incubator systems do not supply or have the ability to balance of gases in the system and the balance of gases within the chambers.

Some problems facing known incubator system include: they do not have a gas monitoring system; they do not recirculate the gases; are not able to constantly provide multiple chambers with the correct percentages of the necessary gases in order to grow embryos and increase the likelihood of a live birth. Another problem with current incubators and incubator systems is there inability to receive the exact gas composition for the incubator environment.

Thus there is a need for an invention that overcomes the above listed and other disadvantages.

### SUMMARY OF THE INVENTION

The disclosed invention relates to a gas recirculation system for an incubated controlled environment, the system comprising: a first mixing box; a second mixing box in fluid communication with the first mixing box; a manifold in fluid communication with the second mixing box; a plurality of incubation chambers in fluid communication with the manifold; a return manifold in fluid communication with the plurality of incubation chambers and in fluid communication with the first mixing box.

The invention also relates to a gas recirculation system for an incubated controlled environment, the system comprising: a first mixing box; a second mixing box in fluid communication with the first mixing box; an inlet port in fluid communication with the second mixing box; a plurality of incubation chamber inlet ports; an incubation chamber in fluid communication with the plurality of incubation chamber inlet ports; a plurality of incubation chamber outlet ports in fluid communication with the incubation chamber; a check valve in fluid communication with the plurality of incubation outlet ports, and in fluid communication with the second mixing box.

In addition, the invention relates to a gas recirculation system for an incubated controlled environment, the system comprising: a pump; a filter in fluid communication with the pump; a sensor box in fluid communication with the filter; a pH sensor box in fluid communication with the sensor box, the pH sensor box comprising a container with embryo culture media and a pH sensor configured to determine the pH of the embryo culture media; a regulator in fluid communication with the pH sensor box; an embryo chamber in fluid communication with the regulator; a second pH sensor box in fluid communication with the regulator, the pH sensor box comprising a container with embryo culture media and a pH sensor configured to determine the pH of the embryo culture media.

The invention also relates to a gas recirculation system for an incubated controlled environment, the system comprising: a means for monitoring the O2 and CO2 concentration of gases in the incubated controlled environment; a means for injecting CO2, N2, and/or O2 into the incubated controlled environment via a mixing box; a means for adjusting the CO2, N2, and O2 levels in the incubated controlled environment.

The invention relates to a gas recirculation system for an incubated controlled environment of claim 16, further comprising: a means for introducing CO2, N2, and/or O2 into the incubated environment in order to maintain about 5% of CO2; about 90% of N2; and/or about 5% of O2 in the incubated environment.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 shows a schematic diagram the gas system device;

FIG. 2 shows a schematic diagram of another embodiment of the invention; and

FIG. 3 shows a schematic diagram of another embodiment of the invention.

### DETAILED DESCRIPTION

The disclosed gas circulation system may provide the following: an enhanced ability to allow better culturing, of the embryos using this system. The disclosed gas circulation system may provide for the long term, uninterrupted culturing of the embryos, within a controlled environment, throughout all growth stages up to re-implantation. The disclosed gas circulation systems allows for a compact apparatus, which can readily adjust and maintain the correct balance of gases within the chambers holding the embryos.

One embodiment of the disclosed gas circulation system may relate to a compact gas system consisting of carbon dioxide (CO2), nitrogen (N2) and oxygen (O2), for the growth of embryos and other biological samples. The disclosed gas circulation system may include a system for regulating the incoming gases, monitoring those gases and adjusting those gases with sensors and valves and the distributing these gases throughout a single and multiple embryo culturing chambers. The disclosed gas circulation system includes compact monitoring devices to assure that the CO2, N2 and O2, levels of the disclosed gas circulation system are suitable and/or optimal for the growth and development of human embryos and properly distributed to one or more incubated chambers. The disclosed gas circulation system may include a system for distributing gases and recirculating the gases, to include filters, pumps, fans and the like. By recirculating the gases within the system and repeatedly purifying these gases; the incubator environment(s) will be far more beneficial for embryo culture and will help conserve the many gases used and the reduced cost of supplying the proper gases to maintain the system. The disclosed gas system may be enclosed in its own enclosure whereby it would be fully operational, may be enclosed in its own chamber and attached to or mounted along with an incubator. By having the system closed and within own enclosure would allow the system to be easily handled, attached, operated and would allow easier maintenance, service and the ability to upgrade the gas system for future changes and upgrades. This gas system will have the ability to easily and readily be attached to an incubator or incubator type device through gas line feeds and the like. This gas recirculation system will greatly improve the consistency of the gases required by the embryos for growth and development, will allow the embryos to grow through to blastocyst stage, increase the cell counts of these embryos and provide a greater likelihood for implantation and the possibility of live birth.

The disclosed gas circulation system may include a system for the introduction of incoming gases, monitoring, adjusting, recirculating, and the continual monitoring of the balance of gases such as carbon dioxide (CO2), nitrogen (N2) and oxygen (O2), inside its incubated environment. The disclosed gas circulation system, in brief, may be a controlled gas system which begins with the introduction of the gases from the incoming gas source, maintains a concise pressure of these gases entering the system, adjusts these gases for the desirable level for these gases, circulates these gases to the chamber, group of chambers or multiple chambers, returns these gases back into a mixing chamber, and then readjusting these gases through a series of sensors and valves achieving a concise balance of these gases within this closed loop gas environment. The disclosed gas circulation system generally improves the regulation of the composition of the gases to be used in the culturing and growth of embryos.

The disclosed gas circulation system may be used in a single culturing environment such as a big-box incubator, smaller multi-compartment incubators, and multiple chambered incubator systems and environments.

The disclosed gas circulation system may maintain the pressure balance of the incoming gases, through regulators, attached to the incoming gas ports. CO2 may be adjusted to a range of about 2% to about 100%, oxygen from about 2% to about 100% and N2 in the range of about 2% to about 100%. The CO2 and N2 may be drawn from incoming gas sources such as cylinders; the oxygen may be drawn from the ambient air, through its own incoming port. The system may use a cylinder of pre-mixed gases of CO2, N2 and O2.

The system may utilize gas regulators for controlling the pressure and amounts of incoming gas to help control and monitor the levels of gas. The disclosed gas circulation system may include pressure sensors, gas sensors and a combination of mixing boxes, blower motor, pumps or fans, a series of tubing to connect the a single compartment, a group of compartments or multiple chambers, used to the incubation and growth of embryos.

The disclosed gas circulation system is unique in that the gas monitoring system may be separate and independent, can easily be plugged into a system of this type and is currently the only system, which will provide monitored, concise flow of the proper amounts of CO2 nitrogen and oxygen to multiple incubation chambers of a system. A unique feature of the system is its ability to maintain the flow of the needed gases at generally all times. This feature is important in that if the power fails and all gases do not reach the embryos they will eventually die. This feature will ensure that the embryos receive, at least CO2, which will keep them going until such time as the system is again activated, or the embryos may be moved to a working incubator.

One embodiment of the invention will control the balance of the gases, such as CO2, N2 and O2 by monitoring the balance of the pH level within a small media or liquid sample which is within a chamber or the system The pH balance of media is a significant factor in the growth of embryos. The pH balance of an embryos culturing media solution is generally about 7.0 to 7.5. This is significant when taking into consideration that the balancing of CO2 at about 5% and N2 at about 90% and O2 at 5%, may not result in the correct pH level within the media for the embryos growth. The ability to read and monitor the pH level will greatly improve the ability for the media to provide the embryo with the correct balance.

The disclosed gas circulation system and the use of this gas circulation system will allow the incubated system and a series of chambers to provide the uninterrupted embryo culturing in this close environment from the retrieval through the blastocyst stag, which may be about 5 to 6 days.

The disclosed gas circulation system and its various embodiments will provide an optimal environment for the growth of embryos and biological specimens.

The disclosed gas circulation system may provide precise amounts of gases, CO2, N2 and O2, for the development of embryos within multiple chambers in incubated environments.

The disclosed gas circulation system, by monitoring the pH within the system and adjusting the gases, CO2, N2 and O2 to maintain a desirable pH level in the enclosed culture media.

The disclosed gas circulation system may provide the precise balance of gases for the optimal uninterrupted, continual growth of embryos of up to about 6 days.

The disclosed gas circulation system may provide precise amounts of gases, CO2, N2 and O2, for the development of embryos within multiple chambers in incubated environments.

The disclosed gas circulation system may provide a method and apparatus for protecting biological specimens such as embryos from outside factors, such as airborne contaminants, disruptive movement, earthquakes and the like.

The disclosed gas circulation system, in one embodiment, may be a multiple gas system, for single chamber incubator environments and multi chambered incubated environments for the uninterrupted culture of human embryos with a combination of gas streams, filters, pumps and a recirculation gas stream. This re-circulated air stream provides a better balance of the needed gases, carbon dioxide, CO2, nitrogen, N2, and oxygen, O2.

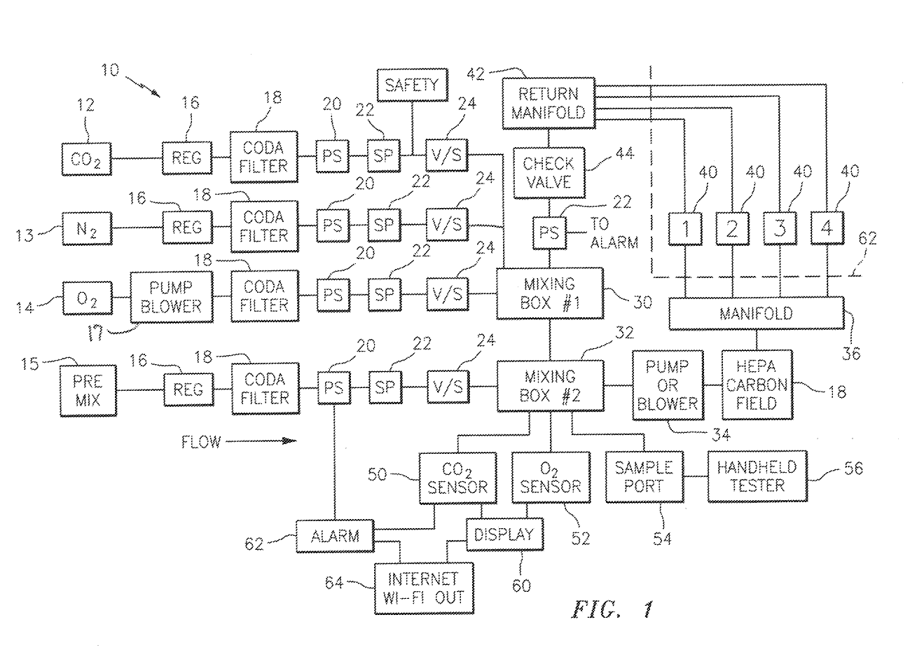

FIG. 1. Shows an embodiment of the disclosed gas circulation system 10. The gas system may provide a well-balanced gas mixture and gas flow for the incubation of human embryos. The system 10 comprises incoming gas from tanks or incoming gas lines, 12, 13, 14. Gas line 12 may be for CO2, gas line 13 may be for N2, and gas line 14 may be for air and/or O2. Lines 12, 13 enter the system through regulators 16, the gas is then passed through a filter, such as a Coda® Inline Filter 18, containing a carbon and a HEPA filter, then to the pressure switch 20, which will report and/or set alarm 62, if any drops in the incoming pressure and empty tanks are detected. The system also comprises small particulate filters 22 in communication with solenoid valve 24, the valves 24 control the amount of gas released into the system.

The small particulate filters 22 are to prevent small debris from entering the solenoid and possibly leading to clogging. The gases then enter the mixing chamber 30, where the CO2 and N2, may be mixed with an incoming air.

The O2 for the system in drawn from the ambient air intake 14, assisted with a pump 17, through a filter, such as a Coda® Inline Filter 18, then a pressure switch 20, a small particulate filter 22, then through a valve or solenoid 24. The gases are pumped for the pressure flow into the system, the filter 18, may contain carbon and/or a potassium permanganate and carbon mixture.

The three gases CO2, N2 and O2 are collected and mixed in mixing box #1, 30. The gases from mixing box #1 then go into a second mixing box #2, 32. At this time the 3 original gases are then mixed with the returning gases from the chambers 40, through the return manifold 42, and check valve 44 and pressure switch 22. Mixer box #2 32, prior to any adjustments should contain a different percentage mixture of gases, as now it includes the gases returning from the chambers, which may contain a different percentage of the 3 gases, sometimes more O2, due to the chamber being open and closed or certain gases being ‘used up’ in the system.

In mixer box #2 32, the combination of air is tested for the percentages of CO2 and O2, by the low a better testing of the levels of CO2 and O2 by sensors 50 and 52. These sensors will send an electronic single to a display screen 60 which will be positioned on the front of the disclosed gas circulation system. The gases will be adjusted by the sensor readings 50 and 52 which will send electronic signals to the solenoid's valves 24, more of the original CO2 and N2 into the system to offset any reduced levels that are introduced through the returning gas lines. The percentage of the CO2 and O2 released into the system can be any suitable percentage. In this embodiment the percentage is 100%.

The mixture of gases that is collected in a mixing box #2 32, allow a better mixing of the gases and the sampling of those gases. The gases than may be pumped, by a pump or fan system 34, through a filter, such as but not limited to a Coda® Inline filter 18, into a manifold 36. This manifold 36 will then distribute the gas mixture evenly through to the four independent incubation chambers 40. In this embodiment, there are four independent chambers as an example for this system. In other embodiments included in this invention, there may be fewer or more chambers. Those chambers 40 will then return those gases to a manifold 42, then through a check valve 44, and then a possible pressure switch 22. This pressure switch will indicate if the system is operating on the return side. This pressure switch 22 may not be in all embodiments.

The gases may be sampled through a sample port 54. This sample port 34 is attached to the mixing box #2 32. The sample may be tested with an exterior testing device, handheld or connected.

The embodiment shows premixed gas 15 entering the system. This gas source has a premix of the gases, CO2, N2 and O2, and may be used. This may be a choice of the user. This premixed gas 15, may be attached to or bypasses mixing tank #1 and go to mixing box #2, and mixed there.

The disclosed gas circulation system has the ability to alert the user to any loses in gas pressure with the pressure switches 24, which will trigger an alarm 62, be shown on the display 60 and may notify the user through a connection device 56, to the internet, cell phone call, text message, WI-FI and the like.

This embodiment may contain a backup system for incoming gases in case of loss of power or being disconnected. There may be an addition set of solenoids, one for CO2, one for N2 and one for O2. These solenoids would activate and open upon the loss of power. They would open and allow the introduction of CO2, N2 and O2, in the correct amounts as needed to maintain the levels of the gases, especially CO2 for the continued safety of the embryos.

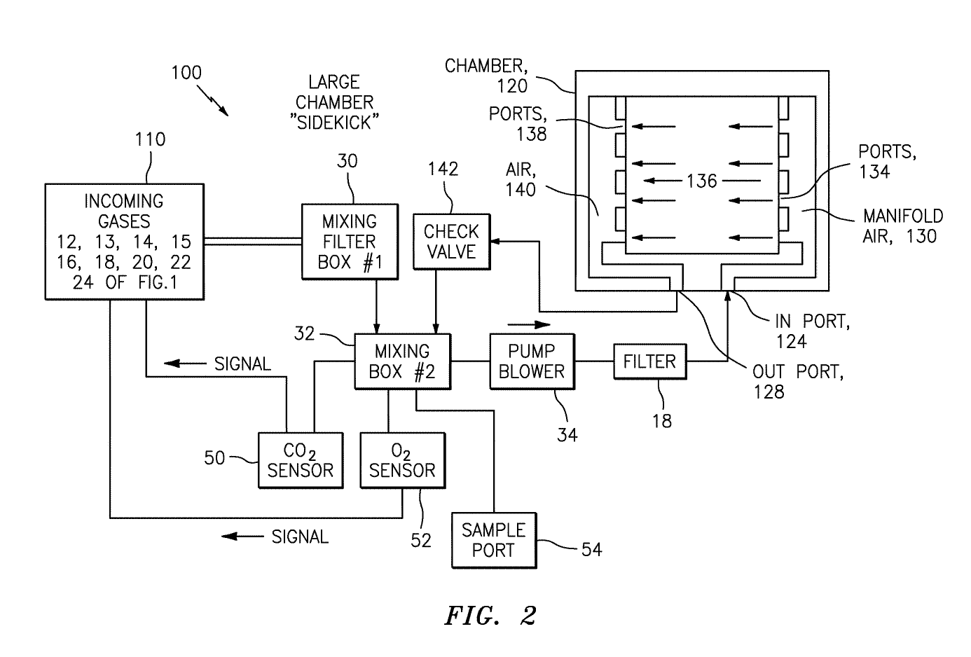

FIG. 2 shows another embodiment of the gas system, which distributes the mixed gases through a series of manifolds within the system.

Some components and gases shown in box 110 indicate items from FIG. 1. From 110 the gases enter mixing box #1 30, then to mixing box #2 32. The gases then pass through a pump, blower or fan 34, through a filter, such as a Coda Inline filter 18. In this embodiment the gases, then go into a passageway 130, through an inlet port 124. The gases then go through a series of openings 134, into a chamber 136. The gases then pass through this chamber 136, and exit through openings 138. This creates a balance and consistent flow of the gases as well as the proper composition of those gases into the chamber 136. The gases exit the chamber through the openings 138, pass thorough the passage way 140 and are drawn from the passage way 140, through the ‘out’ port 128. From the ‘out’ port 128 the gases are then re-circulated back to mixing box #2 32. The returning gases are then mixed with the incoming gas in Box #2, where the CO2 sensor 50 reads the gases, and O2 sensor 52, which will send a signal to the valves located in 110.

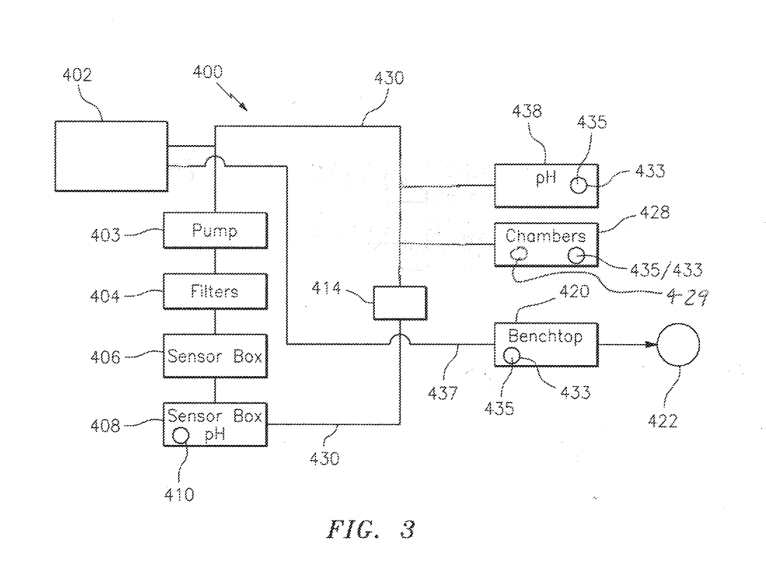

FIG. 3 shows an additional embodiment of the invention, which may be described as an abbreviated version of the embodiments shown in FIG. 1 and/or FIG. 2. The system allows the incoming gases to go through regulators and solenoid valves located in 402, then to a pump 403, and then to filters 404. Then the gases enter a sensor box 406, which in this embodiments contains an O2 and CO2 sensor, which will control the introduction of the gases.

In this embodiment the gas circulation system contains a holding device, such as a petri dish or test tube, which contains an embryo culturing media solution, within one of the chambers or in a separate chamber or may be placed in the main flow of the system. The invention contains a pH-monitoring device, for determining the pH level, within this media solution. This device will continually monitor the pH balance of the system and send a message to the incoming gas system. The invention will use the reading of pH results, within the media solution to balance the gas concentrations within the system, through the introduction of the CO2, N2 or O2. This create a unique system for continually monitoring the gas concentrations introduced and within the system, as this reads the results of the effect of the gas concentrations on the embryo culture media, where the embryo will resides in a similar dish. This embodiment uses pH as an indicator and as opposed to only reading the levels of gases in the system. This will allow the system to create the direct relationship of the gases and their functioning in balancing the pH level of the culture media and the resulting better concentration of the gases.

This embodiment of the invention contains a pH sensor located in 408. This box 408 contains a dish 410, which contains embryo culture media. This media will be changeable and have ease in access in the system configuration. The embodiments contain a second method for pH detection. This may also be in a separated chamber 428, in dish or tube 429. This gives the system the ability to be independent of the incubators using the gases, while being able to monitor the pH of the system, or the pH sensor and dish, containing culture media may be held in an external chamber such as 428.

The internal processor, not shown, will monitor the pH, compare it to the level that the user desires, in this example, within a range of about 7.0 to 7.5, and then transmit electronics signals to the incoming CO2, N2 and O2 solenoid contained in 402 to release the incoming gases, as needed.

In this example, it shows and contains a separate outgoing gas port, regulator or valve 414, by a solenoid or control valve, which will release the gases into open ended system 420, such as that used by some of the current large ‘big box’ incubators, such as those of Forma or benchtop incubators, such as the Cook Minx® and Planar® Benchtop brands. In these type benchtop incubators, they do not have a return loop for the gas. The gases may be held in the incubator until it is replenished by the gas systems, or the incubator may allow the gases to slowly leak out the chamber, or hold the gases until the door or lid is opened, then the incubator is refilled.

This invention is unique in that it may constantly adjust the balance of the CO2, N2 and O2 in the ‘loop’ of the system, then releasing these more concise gasses to these incubators. Currently, they may rely on a premixed gas in a tank or a single, one direction, stream of the gas flow, which has its inherent inconsistencies, and would not consistently contain the optimum balance of gases.

The system will circulate the gases at a rate of about 10 to about 150 liters per hour and then release the gases to these incubators at a lesser rate of flow, which may be less than 2 liters a minute into the benchtop. The gasses released to the incubators 420 may be in a continued flow, which would include an outgoing regulator 414 and tubing to attach the incubators and to supply the gases. The gases will be held in the incubators 420, for the time dictated by the benchtop programming or protocols. In one example the benchtop will hold the same gas in the chamber, until the top is opened again, at which time the system will again fill the chamber with gas, up its closing. A second method is to provide the chambers with a continuous flow of gases, whereby the gases will slowly ‘leak’ out of the chambers and into the atmosphere.

In this example the gas will be released through a valve 414 to the incubator 420 in the incubator or benchtop will be a media dish and solution 433 and a pH monitoring device 435, within this dish. The pH monitoring device will then send a signal via 437 to the incoming gas system 402 which will the adjust the incoming gases to optimize the desired pH level. The system may leak the gases to the atmosphere 422.

The gases may be released to independent chambers 428, similar to what is described in FIG. 1, in chamber 428, or series of chambers. Chamber 428 may contain a pH monitoring system 435 which is with in the dish and media 433. In this embodiment there may be a separate area or chamber where the gases will flow through and which will contain dish 433 and pH monitoring device 435. This monitoring device will continually monitor the pH balance of the gases and reports those gases back to the incoming gas system 402. This separate area holding 438 has the advantage that the pH monitoring device will be included and reside in the system itself and will be able to consistently monitor pH balances and communicate back to the incoming gas electronics 402 so that the gases can be constantly adjusted to optimal levels.

The conventional incubators and benchtop 420, may be retrofitted to include the invention. The invention has the advantage of being able to retrofitted to conventional incubators and benchtops, which are in abundance, allowing these units to include the benefits of the invention.

The disclosed system may maintain and hold these gases for an extended period of time. An embodiment of the invention may be to recirculate the gases, through the benchtop and which are may then be recirculate through the gas mixer of the invention.

The disclosed gas circulation system provides each of the incubator chambers with a consistent flow of well balanced and mixture of the gases needed for the incubation and development of the embryos and specimens. The disclosed gas circulation system gives the embryos a large volume of gases, repeatedly cleaned by the several filters, resulting in a superior environment for the development and possibly improves birth rates.

The disclosed gas circulation system has many advantages. The disclosed system may be standalone or and to gas filtration system, which will consistently provide the precise gas mixtures and may be easily attachable to an incubator, multiple incubators or multiple chambers within an incubator environment. The disclosed system may contain a controlled environment of temperature and gas levels, along with a multiple chambers in order to enhance and grow embryos, stem cells or other biological specimens. The system may repeatedly clear the air and gases in and enclosed environment with greater reduction of particulate, VOC's and CAC's, and aid in the growth of embryos, stem cells and other biological specimens. The system may be enclosed within the incubator itself or to be enclosed within a chamber, which may be inserted into the incubated compartment or may be attached to the incubator to perform. In another embodiment, the gas mixing system may be in its own enclosure, which would then make it a complete system, adaptable for many individual incubators, adaptable for multiple chambers and would be easily replaceable for maintenance, service and upgrades. The disclosed system may provide a controlled environment of incoming CO2, N2 and O2 gas levels, to consistently control the mix of these gases and the percentage of these gases in order to create a much better environment for the growth of embryos, stem cells and or biological specimens. The disclosed system may be an embryo-culturing device, which will greatly reduce the likelihood of service issues, be readily being interchangeable and to be able to be multi configured to suit the needs of the users

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A gas recirculation system for an incubated controlled environment, the system comprising:
a first mixing box;
a second mixing box in fluid communication with the first mixing box;
a manifold in fluid communication with the second mixing box;
a plurality of incubation chambers in fluid communication with the manifold;
a return manifold in fluid communication with the plurality of incubation chambers and in fluid communication with the first mixing box.

2. The gas recirculation system for an incubated controlled environment of claim 1, further comprising:
a means for monitoring the O2 and CO2 concentration of gases in the second mixing box;
a means for injecting CO2, N2, and/or O2 into the first mixing box;
a means for adjusting the CO2, N2, and O2 levels in the second mixing box.

3. The gas recirculation system for an incubated controlled environment of claim 2, further comprising:
a means for introducing CO2, N2, and/or O2 into the first mixing box in order to maintain about 5% of CO2; about 90% of N2; and/or about 5% of O2 in the incubated environment.

4. The gas recirculation system for an incubated controlled environment of claim 1, further comprising:
a CO2 sensor in fluid communication with the second mixing box;
an O2 sensor in fluid communication with the second mixing box;
a sample port in fluid communication with the second mixing box, the sample port configured to receive a tester.

5. The gas recirculation system for an incubated controlled environment of claim 4, further comprising:
a display in signal communication with the CO2 sensor and the O2 sensor;
an alarm in signal communication with the CO2 sensor;
an computer network in signal communication with the display and the alarm.

6. The gas recirculation system for an incubated controlled environment of claim 1, further comprising:
a CO2 supply in fluid communication with a first regulator;
a first filter in fluid communication with the first regulator;
a first pressure switch in fluid communication with the first filter;
a first small particulate filter in fluid communication with the first pressure switch;
a first valve in fluid communication with the first small particulate filter, and in fluid communication with the first mixing box;
an N2 supply in fluid communication with a second regulator;
a second filter in fluid communication with the second regulator;
a second pressure switch in fluid communication with the second filter;
a second small particulate filter in fluid communication with the second pressure switch;
a second valve in fluid communication with the second small particulate filter, and in fluid communication with the first mixing box;
an O2 supply in fluid communication with a third regulator;
a third filter in fluid communication with the third regulator;
a third pressure switch in fluid communication with the third filter;
a third small particulate filter in fluid communication with the third pressure switch;
a third valve in fluid communication with the third small particulate filter, and in fluid communication with the first mixing box;
a premixed gas supply in fluid communication with a fourth regulator;
a fourth filter in fluid communication with the fourth regulator;
a fourth pressure switch in fluid communication with the fourth filter;
a fourth small particulate filter in fluid communication with the fourth pressure switch;
a fourth valve in fluid communication with the fourth small particulate filter, and in fluid communication with the first mixing box.

7. A gas recirculation system for an incubated controlled environment, the system comprising:
a first mixing box;
a second mixing box in fluid communication with the first mixing box;
an inlet port in fluid communication with the second mixing box;
a plurality of incubation chamber inlet ports;
an incubation chamber in fluid communication with the plurality of incubation chamber inlet ports;
a plurality of incubation chamber outlet ports in fluid communication with the incubation chamber;
a check valve in fluid communication with the plurality of incubation outlet ports, and in fluid communication with the second mixing box.

8. The gas recirculation system for an incubated controlled environment of claim 7, further comprising:
a means for monitoring the O2 and CO2 concentration of gases in the second mixing box;
a means for injecting CO2, N2, and/or O2 into the first mixing box;
a means for adjusting the CO2, N2, and O2 levels in the second mixing box.

9. The gas recirculation system for an incubated controlled environment of claim 8, further comprising:
a means for introducing CO2, N2, and/or O2 into the first mixing box in order to maintain about 5% of CO2; about 90% of N2; and/or about 5% of O2 in the incubated environment.

10. The gas recirculation system for an incubated controlled environment of claim 7, further comprising:
a CO2 sensor in fluid communication with the second mixing box;
an O2 sensor in fluid communication with the second mixing box;
a sample port in fluid communication with the second mixing box, the sample port configured to receive a tester.

11. The gas recirculation system for an incubated controlled environment of claim 10, further comprising:
a CO2 supply in fluid communication with a first regulator;
a first filter in fluid communication with the first regulator;
a first pressure switch in fluid communication with the first filter;
a first small particulate filter in fluid communication with the first pressure switch;
a first valve in fluid communication with the first small particulate filter, and in fluid communication with the first mixing box, the first valve in signal communication with the CO2 sensor and the O2 sensor;
an N2 supply in fluid communication with a second regulator;
a second filter in fluid communication with the second regulator;
a second pressure switch in fluid communication with the second filter;
a second small particulate filter in fluid communication with the second pressure switch;
a second valve in fluid communication with the second small particulate filter, and in fluid communication with the first mixing box, the second valve in signal communication with the CO2 sensor and the O2 sensor;
an O2 supply in fluid communication with a third regulator;
a third filter in fluid communication with the third regulator;
a third pressure switch in fluid communication with the third filter;
a third small particulate filter in fluid communication with the third pressure switch;
a third valve in fluid communication with the third small particulate filter, and in fluid communication with the first mixing box, the third valve in signal communication with the CO2 sensor and the O2 sensor;
a premixed gas supply in fluid communication with a fourth regulator;
a fourth filter in fluid communication with the fourth regulator;
a fourth pressure switch in fluid communication with the fourth filter;
a fourth small particulate filter in fluid communication with the fourth pressure switch;
a fourth valve in fluid communication with the fourth small particulate filter, and in fluid communication with the first mixing box, the fourth valve in signal communication with the CO2 sensor and the O2 sensor.

12. A gas recirculation system for an incubated controlled environment, the system comprising:
a pump;
a filter in fluid communication with the pump;
a sensor box in fluid communication with the filter;
a pH sensor box in fluid communication with the sensor box, the pH sensor box comprising a container with embryo culture media and a pH sensor configured to determine the pH of the embryo culture media;
a regulator in fluid communication with the pH sensor box;
an embryo chamber in fluid communication with the regulator;
a second pH sensor box in fluid communication with the regulator, the pH sensor box comprising a container with embryo culture media and a pH sensor configured to determine the pH of the embryo culture media.

13. The gas recirculation system for an incubated controlled environment of claim 12, further comprising:
a means for monitoring the O2 and CO2 concentration of gases in the sensor box;
a means for injecting CO2, N2, and/or O2 into the sensor box;
a means for adjusting the CO2, N2, and O2 levels in the sensor box.

14. The gas recirculation system for an incubated controlled environment of claim 13, further comprising:
a means for introducing CO2, N2, and/or O2 into the sensor box in order to maintain about 5% of CO2; about 90% of N2; and/or about 5% of O2 in the incubated environment.

15. The gas recirculation system for an incubated controlled environment of claim 12, further comprising:
a CO2 supply in fluid communication with a first regulator;
a first filter in fluid communication with the first regulator;
a first pressure switch in fluid communication with the first filter;
a first small particulate filter in fluid communication with the first pressure switch;
a first valve in fluid communication with the first small particulate filter, and in fluid communication with the pump;
an N2 supply in fluid communication with a second regulator;
a second filter in fluid communication with the second regulator;
a second pressure switch in fluid communication with the second filter;
a second small particulate filter in fluid communication with the second pressure switch;
a second valve in fluid communication with the second small particulate filter, and in fluid communication with the pump;
an O2 supply in fluid communication with a third regulator;
a third filter in fluid communication with the third regulator;
a third pressure switch in fluid communication with the third filter;
a third small particulate filter in fluid communication with the third pressure switch;
a third valve in fluid communication with the third small particulate filter, and in fluid communication with the pump;
a premixed gas supply in fluid communication with a fourth regulator;
a fourth filter in fluid communication with the fourth regulator;
a fourth pressure switch in fluid communication with the fourth filter;
a fourth small particulate filter in fluid communication with the fourth pressure switch;
a fourth valve in fluid communication with the fourth small particulate filter, and in fluid communication with the pump;
a benchtop embryo incubator in fluid communication with the first valve, second valve, third valve, and fourth valve; and
a gas output to atmosphere in fluid communication with the benchtop embryo incubator.

16. A gas recirculation system for an incubated controlled environment, the system comprising:
a means for monitoring the O2 and CO2 concentration of gases in the incubated controlled environment;
a means for injecting CO2, N2, and/or O2 into the incubated controlled environment via a mixing box;
a means for adjusting the CO2, N2, and O2 levels in the incubated controlled environment.

17. The gas recirculation system for an incubated controlled environment of claim 16, further comprising:
a means for introducing CO2, N2, and/or O2 into the incubated environment in order to maintain about 5% of CO2; about 90% of N2; and/or about 5% of O2 in the incubated environment.
